(19) United States
(12) Patent Application Publication
(10) Pub. No.: US 2021/0138002 A1
(43) Pub. Date: May 13, 2021

(54) PREPARATION FOR THE TREATMENT OF INFLAMMATORY BOWEL DISEASE USING A WHOLE PLANT FIBRE EXTRACT FROM SUGARCANE

(71) Applicant: UNIVERSITY OF TASMANIA, Sandy Bay, Tasmania (AU)

(72) Inventors: Rajamaran Eri, Brisbane, Queensland (AU); Gordon Edwards, Brisbane, Queensland (AU); Kent John Neil Taylor, Brisbane, Queensland (AU); Roger Anthony Stanley, Brisbane, Queensland (AU); Tanvi Sandesh Shinde, Brisbane, Queensland (AU)

(21) Appl. No.: 17/251,135

(22) PCT Filed: Jun. 12, 2019
(86) PCT No.: PCT/AU2019/050604

§ 371 Date: Dec. 10, 2020

(30) Foreign Application Priority Data
Jun. 15, 2018 (AU) 2018902145

(51) Int. Cl. A61K 35/742; A23L 33/125; A23L 33/135; A23L 33/22; A23L 2/52; A61K 36/899

(52) U.S. Cl. CPC A61K 35/742; A23L 33/125; A23L 33/135; A61K 2035/115; A23L 2/52; A61K 36/899; A23L 33/22

(57) ABSTRACT

The invention relates to the field of food supplement manufacture and therapeutic manufacture. In particular, the invention relates to use of a prebiotic whole plant fibre extract from sugarcane in the diet of an individual for the prophylaxis and/or treatment of Inflammatory Bowel Disease. The invention also relates to a combination of the prebiotic whole plant fibre extract from sugarcane and probiotic strains, the synbiotic use of said combination in the diet of an individual, and the improved outcomes of the synbiotic approach in the prophylaxis and/or treatment of Inflammatory Bowel Disease.

### TECHNICAL FIELD

The invention relates to the field of food supplement manufacture and therapeutic manufacture. In particular, the invention relates to use of a prebiotic whole plant fibre extract from sugarcane in the diet of an individual for the prophylaxis and/or treatment of Inflammatory Bowel Disease. The invention also relates to a combination of the prebiotic whole plant fibre extract from sugarcane and probiotic strains, the synbiotic use of said combination in the diet of an individual, and the improved outcomes of the synbiotic approach in the prophylaxis and/or treatment of Inflammatory Bowel Disease.

### BACKGROUND OF THE INVENTION

Inflammatory Bowel Disease (IBD) is the collective condition label given to a number of gastrointestinal (GI) disorders that result in colitis of the GI tract, or chronic gut inflammation. Colitis refers to the inflammation of the inner lining of the colon (and in some cases may involve the rectum), and there are numerous causes of colitis including Ulcerative Colitis (UC), Ischemic colitis, microscopic colitis, Crohn's disease, and allergic reactions (including food sensitivities). Commonly associated inflammatory bowel effects or results of treatment can be pouchitis, diverticulitis, and the inflammation linked to antibiotic-associated diarrhea.

IBD is now considered an emerging global disease with increasing prevalence, severity and complexity especially in western societies. An increasing trend is the age of onset with more young patients encountering IBD symptoms than previously recorded. The variation of symptoms can be significant and unpredictable in onset, trigger patterns, severity for each individual with the adverse side effects of treatment leading to the broader concerns of an individual's mental, and social wellbeing. Studies are now correlating incidence of IBD to increased risks of colorectal cancer.

A state of chronic gut inflammation involves a dysregulated mucosal immune response against commensal gut microbes in genetically susceptible individuals with impaired gut epithelial integrity. Several studies report dysbiosis with lower intestinal microbial diversity and altered microbial balance in colitis patients compared to healthy individuals. In addition, increased gut permeability owing to weakened intestinal epithelial barrier integrity is a prominent feature in colitis. In this context, probiotic and prebiotic components that can influence gut microbiota, strengthen intestinal barrier integrity, and modulate immune response, provide strategies to attenuate intestinal inflammation.

Treatments of IBD are wide and varied involving pharmaceuticals and in some cases surgery. The baseline treatment and avoidance of inflammatory events can be approached from a dietary perspective and studies demonstrate a role for prebiotics, probiotics and the synergistic combination administration, labelled synbiotics.

Synbiotics, being a combination of prebiotic and probiotic ingredients, can potentially offer prophylactic and therapeutic effects that could function synergistically to confer health benefits to the host. Administration of probiotics relies on an optimal viability of microbes or by-products. In search of higher viability microbes, spore-producing probiotics have become a growing research focus owing to their ability to survive the gastric transit, harsh manufacturing and storage temperatures. Additionally, many prebiotics are often purified fibres of limited chemical complexity and do not present the multiple range of glycosidic bond types that naturally occur in fruit and vegetables.

Developing prebiotics that replicate nature's complexity of chemical structures has not been a standard practise in commercially available products to date. However, providing a simple prebiotic that is readily digestible may not be the correct strategy given it could result in a natural selection towards bacteria that thrive on the simplified fibre structures and unbalance the diversity that is known to be an important factor in optimal function of the bowel and protection from disease.

A unique prebiotic phytonutrient fibre extract from sugarcane, manufactured to preserve the cell wall components provides a more representative cellular fibre component from plant dietary sources. While sugarcane fibre has historically been labelled as an insoluble fibre, improved fibre analysis techniques and studies into the fermentability of sugarcane fibrous matter reveals digestibility of the fibrous sub-structures displaying insoluble fibre characteristics by bacteria, specifically those that reside and can function in the host colon. Providing this complex and more nature equivalent nutrient source to the microorganisms of the colon, including functional delivery of sugarcane's characterised antioxidant bioactives (phenolics, flavonoids, and polycosanols), delivers a positive pressure on maintaining or increasing microbial diversity, rather than the reduction of diversity with limited complexity prebiotics.

By targeting the normalisation of gut microbiota, the production of beneficial by-products of microbial digestion (such as short chain fatty acids), the delivery of plant antioxidants to the colon, and supplying a known beneficial probiotic, the synergistic effects can be optimised beyond the sum of its parts.

Many of the conditions and symptoms described in humans also have their counterparts in veterinary and animal agriculture. Intestinal inflammation is common in domestic animals (dogs, cats, mice, rabbits and horses) as well as captive-bred animals (chickens, cattle, goats, and fish). It is a logical conclusion that these animals would respond to similar prophylactic treatments to reduce the inflammation state of their bowels and result in similar health improvements for companion animals and deliver improved quality of life and economic benefits to industry.

Accordingly, it is an object of this invention to provide use of a prebiotic whole plant fibre extract from sugarcane in the diet of an individual for the prophylaxis and/or treatment of Inflammatory Bowel Disease.

An alternative objection of the invention is to provide use of a prebiotic whole plant fibre extract from sugarcane in the diet of an individual for the ameliorating the effects of Inflammatory Bowel Disease.

A further alternative object of the invention is to provide a synergistic combination of a prebiotic whole plant fibre extract from sugarcane and probiotics that ameliorates the symptoms of Inflammatory Bowel Disease, as a long-term prophylactic treatment by inclusion into the regular diet.

An even further alternative objection of the invention is to provide a synergistic combination of a prebiotic whole plant fibre extract from sugarcane and probiotics that ameliorates the inflammatory states of Inflammatory Bowel Disease, as a long-term prophylactic treatment by inclusion into the regular diet.

The reference to any prior art in this specification is not, and should not be taken as an acknowledgement or any form of suggestion that the prior art forms part of the common general knowledge in the art in Australia or in any other country.

### SUMMARY OF THE INVENTION

The present invention is directed to use of a prebiotic whole plant fibre extract from sugarcane in the diet of an individual for the prophylaxis and/or treatment of inflammatory bowel disease.

The invention is also directed to a combination of the prebiotic whole plant fibre extract from sugarcane and probiotic strains, and the synbiotic use of said combination in the diet of an individual for the prophylaxis and/or treatment of inflammatory bowel disease.

According to a first aspect of the invention, there is provided use of a prebiotic phytonutrient fibre material extracted from sugarcane for prophylaxis and/or treatment of the effects of inflammatory bowel disease.

It is particularly preferred that the prebiotic phytonutrient fibre material extracted from sugarcane is KfibreTm.

According to a second aspect of the invention, there is provided a composition comprising a prebiotic phytonutrient fibre material extracted from sugarcane, and at least one probiotic bacterial strain.

Preferably, the prebiotic phytonutrient fibre material is prepared via a process including the steps of: subjecting sugarcane to at least one wet diffusion step to separate sugars from a residual fibre material whilst maintaining nutrient content; and subjecting the residual fibre material to a rapid, low-heat drying process to retain biologically active molecules in the fibre, and to enhance the water retention properties of said residual fibre product. Preferably, the wet diffusion step is a diffusion extraction, performed under relatively low-shear conditions. Preferably, the wet diffusion step is performed within the temperature range 25° C. to 70° C.

The composition can further comprise a pharmaceutically acceptable carrier, solvent, base or excipient.

There are a number of advantages to using prebiotic fibre material extracted from sugarcane in the way described above. Firstly, there are no adverse allergic effects associated with this source. Secondly, as a whole of plant fibre source it more accurately represents whole vegetables than fibre sources generated from grains or chemically manufactured from other sources. Thirdly, it is high in essential micronutrients and has the ability to protect micronutrients from other foods when consumed in conjunction with a meal.

Sugarcane fibre prepared using the steps described above also has several advantageous properties compared to incomplete (not whole) plant fibres such as bran, psyllium husk and inulin. The fibre prepared from sugarcane is a true lignose, hemicellulose and cellulose combination, like the total dietary fibres found in most vegetables. Pectin is a significant component (about 10%) of cell walls, and being a soluble, metabolizable fibre, could also play a role in the activity of the sugarcane fibre as a prebiotic. Other components of the sugarcane fibre include xylan (which is soluble) and arabinoxylan polymers. The sugarcane fibre can be classed as almost entirely insoluble fibre, using the standard chemical methods of classification, however, it has many of the properties of soluble fibres such as high water-binding capacity (up to 8-10 times by weight) and a prebiotic effect.

This combination of both soluble and insoluble fibre characteristics and the essentially “whole food” nature of the sugarcane fibre most likely allows for a food profile that more closely mimics an ideal diet.

The at least one probiotic bacterial strain can be commercially sourced from bacterial strains that are known and correlated to the reduction of chronic gut inflammation or to the support of the normal function of the gastrointestinal tract. Preferably, the at least one probiotic bacterial strain is spore-forming, thereby providing robustness to survive and retaining viability in the colon of a host. Alternatively, the at least one probiotic bacterial strain can be heat stable to ensure viability during manufacture. In a further alternative, the at least one bacterial strain can be acid stable, allowing the at least one bacterial strain to remain viable in the host colon after administration. In certain embodiments, the at least one probiotic bacterial strain is spore-forming heat stable and acid stable.

Preferably, the at least one probiotic bacterial strain is selected from the group consisting of: Bacillus coagulans, Lactobacillis (L. plantarum, L. paracasei, L. acidophilus, L. casei, L.rhamnosus, L. crispatus, L. gasser L. reuteri, L. bulgaricus), Bifidobacterium (B. longum, B. catenulatum , B. breve, B. animalis, B. bifidum), Streptococcus (S. sanguis, S. oralis, S. mitis, S. thermophilus, S. salivarius), Bacillus (B. subtilis, B. laterosporus), Lactococcus (L. lactis), Enterococcus (E. faecium), Pediococcus (P. acidilactici), Propionibacterium (P. jensenii, P. freudenreichii), Peptostreptococcus (P. productus), and Saccharomyces (S. boulardii).

According to a third aspect of the invention, there is provided use of a food product comprising a prebiotic phytonutrient fibre material extracted from sugarcane, for prophylaxis and/or treatment of the effects of inflammatory bowel disease.

According to a fourth aspect of the invention, there is provided a food product comprising a prebiotic phytonutrient fibre material extracted from sugarcane, and at least one probiotic bacterial strain.

According to a fifth aspect of the invention, there is provided use of a composition comprising a prebiotic phytonutrient fibre material extracted from sugarcane, for prophylaxis and/or treatment of the effects of inflammatory bowel disease.

According to a sixth aspect of the invention, there is provided use of a composition comprising a prebiotic phytonutrient fibre material extracted from sugarcane, for ameliorating the effects of inflammatory bowel disease.

According to a seventh aspect of the invention, there is provided a composition for ameliorating the effects of Inflammatory Bowel Disease; said composition comprising prebiotic fibre material extracted from sugarcane, and at least one probiotic bacterial strain.

According to an eighth aspect of the invention, there is provided use of a food product for ameliorating the effects of Inflammatory Bowel Disease; said food product comprising prebiotic fibre material extracted from sugarcane.

According to a ninth aspect of the invention, there is provided a food product for ameliorating the effects of Inflammatory Bowel Disease; said food product comprising prebiotic fibre material extracted from sugarcane, and at least one probiotic bacterial strain.

The food product can be utilized in the diet for a defined period of time for ameliorating the effects of IBD. Alternatively, the food product can be utilized in the diet over a prolonged period of time, including indefinitely, to ameliorate the effects of IBD and/or as a prophylactic for IBD.

According to a tenth aspect of the invention, there is provided use of a prebiotic phytonutrient fibre material extracted from sugarcane, in the manufacture of a therapeutic for ameliorating the symptoms and/or inflammatory states of Inflammatory Bowel Disease, or for providing a prophylactic effect.

According to an eleventh aspect of the invention, there is provided use of a prebiotic phytonutrient fibre material extracted from sugarcane, and at least one probiotic bacterial strain, in the manufacture of a therapeutic for ameliorating the symptoms and/or inflammatory states of Inflammatory Bowel Disease, or for providing a prophylactic effect.

According to a twelfth aspect of the invention, there is provided use of a prebiotic phytonutrient fibre material extracted from sugarcane, in the manufacture of a food product for ameliorating the symptoms and/or inflammatory states of Inflammatory Bowel Disease, or for providing a prophylactic effect.

According to a thirteenth aspect of the invention, there is provided use of a prebiotic phytonutrient fibre material extracted from sugarcane, and at least one probiotic bacterial strain, in the manufacture of a food product for ameliorating the symptoms and/or inflammatory states of Inflammatory Bowel Disease, or for providing a prophylactic effect.

According to a fourteenth aspect of the invention, there is provided a composition comprising a prebiotic phytonutrient fibre material extracted from sugarcane, and at least one postbiotic.

In the context of this invention, a postbiotic is a non-viable bacterial product or metabolic byproduct from probiotic microorganisms that has biologic activity in the host. In other words, a postbiotic is a fragment or metabolite of a probiotic.

The composition can further comprise a pharmaceutically acceptable carrier, solvent, base or excipient.

The at least one postbiotic can be commercially sourced. Preferably, the at least one postbiotic is sufficiently robust to survive in the colon. Alternatively, the at least one postbiotic can be heat stable to ensure viability during manufacture. In a further alternative, the at least one postbiotic can be acid stable, allowing the at least one postbiotic to remain viable in the colon after administration. In certain embodiments, the at least one postbiotic is heat stable and acid stable.

The at least one postbiotic can be selected from the group consisting of short-chain fatty acids, antimicrobial peptides, nutrients (including amino acids and vitamins such as vitamin K and B-vitamins), carbohydrate-active enzymes.

According to a fifteenth aspect of the invention, there is provided a food product comprising a prebiotic phytonutrient fibre material extracted from sugarcane, and at least one postbiotic.

According to a sixteenth aspect of the invention, there is provided a composition for ameliorating the effects of Inflammatory Bowel Disease; said composition comprising prebiotic fibre material extracted from sugarcane, and at least one postbiotic.

According to a seventeenth aspect of the invention, there is provided a food product for ameliorating the effects of Inflammatory Bowel Disease; said food product comprising prebiotic fibre material extracted from sugarcane, and at least one postbiotic.

The food product can be utilized in the diet for a defined period of time for ameliorating the effects of IBD. Alternatively, the food product can be utilized in the diet over a prolonged period of time, including indefinitely, to ameliorate the effects of IBD and/or as a prophylactic for IBD.

According to an eighteenth aspect of the invention, there is provided use of a prebiotic phytonutrient fibre material extracted from sugarcane, and at least one postbiotic, in the manufacture of a therapeutic for ameliorating the symptoms and/or inflammatory states of Inflammatory Bowel Disease, or for providing a prophylactic effect.

According to a nineteenth aspect of the invention, there is provided use of a prebiotic phytonutrient fibre material extracted from sugarcane, and at least one postbiotic, in the manufacture of a food product for ameliorating the symptoms and/or inflammatory states of Inflammatory Bowel Disease, or for providing a prophylactic effect.

According to a twentieth aspect of the invention, there is provided a method of treating or ameliorating the effects of Inflammatory Bowel Disease in a subject, the method comprising administering to the subject a composition provided by the second or fourteenth aspects, or a food product provided by the fourth or fifteenth aspects.

Throughout this specification, unless the context requires otherwise, the words “comprise”, “comprises” and “comprising” will be understood to imply the inclusion of a stated integer or group of integers but not the exclusion of any other integer or group of integers.

Any of the features described herein can be combined in any combination with any one or more of the other features described herein within the scope of the invention.

### BRIEF DESCRIPTION OF DRAWINGS

FIG. 1 is a diagrammatic representation of the groups of mice used in the comparative trials.

FIG. 2 is a graph showing the effect of the prebiotic on % body weight as an indicator of colitis-associated weight loss.

FIG. 3 is a graph showing the effect of the prebiotic on the Disease Activity Index (DAI).

FIGS. 4A-4C are graphs of the effect of the prebiotic on physiological parameters. 4A: spleen weight; 4B: colon weight; 4C: colon weight:body weight ratio.

FIG. 5A is a graph showing the effect of the prebiotic on colon length. FIG. 5B is a photograph of sample colons.

FIG. 6 shows graphs of the immunomodulatory effects of the prebiotic on IL-1a and IL-1b in the proximal colon and distal colon.

FIG. 7 shows graphs of the immunomodulatory effects of the prebiotic on IL-6 and IL-12 in the proximal colon and distal colon.

FIG. 8 shows graphs of the immunomodulatory effects of the prebiotic on TNF-a and IFN-g in the proximal colon and distal colon.

FIG. 9 shows graphs of the immunomodulatory effects of the prebiotic on IL-1b, IL-10 and IL-12 in serum.

Now will be described, by way of particular, non-limiting examples, preferred embodiments of the invention.

### DETAILED DESCRIPTION OF THE INVENTION

The current invention takes advantage of the properties of a prebiotic fibre isolate produced from sugarcane, in such a way that maximises retention and minimizes destruction of the bioactive molecules. The present inventors have surprisingly found that such a prebiotic fibre isolate is effective in the prophylaxis and/or treatment of inflammatory states associated with colitis. It has also been surprisingly found that combining the prebiotic fibre isolate with a probiotic or a postbiotic results in a synbiotic effect, whereby inflammatory states are reduced by more than when either prebiotic, probiotic, or postbiotic is used in isolation, and more than just the additive effect of the prebiotic and probiotic, or the prebiotic and postbiotic.

The method of preparation of the prebiotic fibre material from sugarcane is broadly similar to that described in the international patent publication no. WO 2011/035381 by KFSU Pty Ltd, which is incorporated herein by reference. However, the method according to the present invention can be defined as having the following essential steps:

1. A sugarcane size reduction step;

2. A relatively ‘gentle’ aqueous extraction step that separates the fibre from other sugarcane fractions, including the sugar fraction, without causing degradation of the fibre functionality; and

3. A relatively gentle drying step that minimises degradation of the fibre functionality.

It is preferred that the aqueous extraction step be an aqueous diffusion extraction performed at about neutral pH. It is also preferred that the drying step be a rapid vortex drying operation that can be achieved via a low temperature vortex dryer, said dryer being able to reduce the wet weight of the sugarcane material from 40-80% wet weight to less than 10% wet weight in 10-30 seconds while not heating the material to a level that would significantly damage the bio-actives in the plant material.

The invention provides for the use of a prebiotic fibre extract from sugarcane, in the formulation of foods, diets or therapeutics that reduces the risk of development of Inflammatory Bowel Disease conditions, or which ameliorates the symptoms of those conditions.

The invention also provides for the use of a synbiotic, namely, a prebiotic fibre extract from sugarcane combined with selected probiotics or postbiotics, in the formulation of foods, diets or therapeutics that reduces the risk of development of Inflammatory

Bowel Disease conditions, or which ameliorates the symptoms of those conditions.

When prepared according to the invention, the synbiotic combination has a number of advantages over other fibre sources and food, including that:
It is relatively hypoallergenic;
It contains both insoluble and bound soluble fibre in beneficial proportions for dietary intake;
It can be prepared in a ‘chemical-free’ manner and contains no harmful trace elements, unlike fibre from other sources such as chemically modified starch;
It can be prepared in such a way as to retain the micronutrients and active molecules found in the “molasses” component of sugarcane, without the need to extract and purify those components for their biological function;
It can be prepared in such a manner to optimize the bioactivity of the prebiotic and also maximise viability of the probiotic or its byproducts (postbiotics).

It is also known that too much fibre in the diet can have several negative side effects including but not limited to constipation, diarrhoea and bad flatulence. Advantageously, in embodiments of the present invention, where the fibre product is added as a cellular based fibre supplement to a subject's diet, dietary fibre intake can be more easily controlled with a sterically hindered restricted rate of microbial metabolism.

The embodiments of the invention can take a number of forms, each with at least one advantage.

As used herein:
A ‘synbiotic’ is a combination of prebiotic whole plant fibre extract from sugarcane and a probiotic bacterial strain or postbiotic byproduct for use in all subsequent formats, formulations and purposes. The synbiotic in most cases, includes but is not limited to, a combination of dry prebiotic and dry probiotic spores, lyophilized or live stabilised bacteria. The synbiotic can also be the combination in an aqueous solution for use in food manufacturing procedures.
A ‘carrier’ is a palatable substrate for the sugarcane fibre, which may or may not contain protein or other nutrients. The carrier can be in a solid or liquid form, including but not limited to: fruit extracts, broths, purees, dairy products, baked goods.
‘Inert filler’ is any product used to increase the bulk of fibre according to the invention to allow for ease of handling by the user. The filler may contain flavours or nutrients, and other dietary fibres to improve mouth feel, but does not necessarily contribute to the total benefit provided by the invention.
‘Pellet’ includes any compact form of the invention, including but not limited to:
A dried pill or tablet in the manner of a vitamin.
A ‘soft lolly’ style lozenge that may be used as a treat or as an addition to other foods

### EXAMPLE 1

In this example, 0.5-5.0 g of a synbiotic according to the invention is pressed into a pellet or added to a flavouring medium and pressed into a pellet. Due to the inherent stability of the prebiotic and selected probiotics, the synbiotic pellets are prepared at a formulation level such that the dose may be varied according to a patient's requirements to reduce the symptoms of Inflammatory Bowel Disease without incurring negative effects.

### EXAMPLE 2

In this example a synbiotic is mixed with a flavoured drink (for example a non-acidic fruit juice or milk) and pasteurised for sterility (1-5 g per 100-250 mL). A drink prepared in this manner is a convenient, ready-to-consume product for the amelioration of chronic IBD symptoms.

### EXAMPLE 3

In this example, a supplement is prepared as an easy-to-measure powder with or without flavours, stabilisers and inert filler, formulated specifically to be combined with water. Specifically, a synbiotic can be mixed with a dry flavour component and an inert filler to form easy-to-use granules. The dose (1-5 g) can be in a convenient single-serve sachet or in a multi-dose bulk pack. The resultant supplement is best suited to allow reduced meal size, as the granules can be mixed with water (thereby allowing less food to be consumed each meal).

### EXAMPLE 4

In this example a synbiotic is prepared in a solid flavoured meal such as a biscuit, a bar, or a bread (baked) product (0.5-5 g per serve of ready mixed food). Multiple biscuits, bars or bread products can be consumed by an individual to provide a specific dosing regimen and optimize compliance of the treatment. This has two advantages over other delivery systems in that it feels more like a treat for the consumer, it eliminates the need for liquid, and reduces the total volume of the stomach contents (a factor for inflammatory bowel conditions). Additionally, the increased saliva production may have a complementary effect with the synbiotic benefits.

### EXAMPLE 5

In this example, a trial involving an established gut inflammation mouse model, was undertaken to measure the efficacy of reducing inflammation responses (employing chemical induction of colitis) of the prebiotic fibre extract from sugarcane. A control was included for baseline comparison.

Prebiotic plant fibre (Kfibre™, sucrose reduced sugar cane fibre), was fed to C57BL/6J strain of inbred mice for 7 days prior to colitis induction with dextran sodium sulfate (DSS). Administration of prebiotic continued for another 7 days along with DSS. Colitis severity was assessed using Disease Activity Index (DAI). Colon samples were collected 7 days after colitis induction for histology, cytokines, myeloperoxidase (MPO) and inducible Nitric Oxide Synthase (iNOS) assay. Spleen weights, as well as colon weight/length and colon weight/body weight ratios, were calculated as macroscopic markers of inflammation. Fecal, mucosal and cecal contents were collected for metabolite, short chain fatty acids (SCFA), and microbial diversity analysis.

The methodology of exposure is shown in FIG. 1. Specifically, three groups of C57BL/6J mice were set up as follows:

Group 1—healthy control group

This group was fed normal chow and autoclaved tap water for the duration of the trial (14 days)

Group 2—DSS control group

This group was fed normal chow for the duration of the trial (14 days), autoclaved tap water for the first 7 days, and then a solution of 2% DSS in water for the next 7 days to induce colitis

Group 3—Kfibre group

This group was fed chow supplemented with Kfibre for the duration of the trial (14 days), autoclaved tap water for the first 7 days, and then a solution of 2% DSS in water for the next 7 days

In vivo analysis was carried out for disease activity index (DAI) [weight loss, stool consistency and occult blood], physiological parameters [spleen weight, colon weight, colon length], immunoassays [quantification of pro-and anti-inflammatory cytokine levels], histopathological evaluation—[H&E staining, IHC analysis], gut microbiota analysis—bacterial 16S rRNA gene sequencing, metabolomics—quantification of SCFA (short chain fatty acids).

The results are set out in FIGS. 2-9.

The effect of the prebiotic on percentage body weight as an indicator of colitis-associated weight loss is shown in FIG. 2. Specifically, FIG. 2 shows that whilst the mice of the DSS control group (with induced colitis) exhibited increased weight loss over the period on DSS, the mice in the Kfibre group exhibited similar weight gain to the mice of the healthy control group. Therefore indicating that the prebiotic was significantly effective in preventing colitis-associated weight loss.

The effect of the prebiotic on the Disease Activity Index (DAI) is shown in FIG. 3. Specifically, the changes in DAI (a cumulative score of weight loss, stool consistency and occult blood) are graphed against the number of days on DSS (chemical induction of colitis). Subjects fed normal chow and water (Group 1) presented no change from baseline on the DAI scale. In contrast, the effect of the DSS chemical colitis inducer (Group 2) raised the inflammation to a DAI average of 5.8, a significantly inflamed state that would be both symptomatic and chronically harmful. The subjects that were administered DSS+prebiotic Kfibre (Group 3) had a reduced inflammation state with a DAI of 2.7 (53% reduction in inflammation, a significant reduction. This represents a clear result that the prebiotic is capable of significant attenuation of colitis.

From FIGS. 4A-4C, it can be seen that the prebiotic significantly reduced spleen weight, reduced colon weight (but statistically insignificant), and reduced the colon weight:body weight ratio significantly, when compared to the DSS control group.

The data in FIG. 5A illustrate that the DSS control group (within induced colitis) showed a significant reduction in colon length, in contrast to the Kfibre group, where the prebiotic significantly reduced colon shortening. Photographs of a sample colon from each group are shown in FIG. 5B.

The immunomodulatory effects of the prebiotic on various cytokines as indicators of inflammation in the proximal colon, distal colon and serum are shown graphically in FIGS. 6-9.

In summary, preconditioning of mice with the prebiotic prior to DSS-induction (Group 3), reduced the severity of the disease symptoms compared with the control-DSS-colitic group (Group 2). Prebiotic fibre (Group 3) improved the DAI compared with that of DSS-colitic mice (Group 2). Unlike the control-DSS group, treatment with the prebiotic proved effective in preventing body weight loss and providing enhanced stool consistency and occult blood score. The treatment effectively improved or eliminated the macroscopic markers of inflammation. Furthermore, in distal and proximal colons, the prebiotic proved significantly effective in modulating pro-inflammatory cytokines.

The prebiotic is thus effective in reducing the severity of chemically-induced colitis. Furthermore, precondition with the prebiotic prior to DSS-induced colitis, reduces the severity of symptoms. The prebiotic also displays significant immunomodulatory ability.

1-29. (canceled)

30. A method for the prophylaxis or treatment of the effects, symptoms or inflammatory states of inflammatory bowel disease in a subject, the method comprising administering to the subject a prebiotic phytonutrient fibre material extracted from sugarcane.

31. The method of claim 30, wherein the prebiotic phytonutrient fibre material is administered in a form selected from the group consisting of a composition, pellet, food product, and beverage.

32. The method of claim 30, further comprising the step of administering to the subject at least one probiotic bacterial strain.

33. The method of claim 32, wherein said at least one probiotic bacterial strain is administered to the subject as spores.

34. The method of claim 30, further comprising the step of administering to the subject at least one postbiotic.

35. The method of claim 30, wherein said prebiotic phytonutrient fibre material is sucrose-reduced sugarcane fibre.

36. The method of claim 30, wherein said prebiotic phytonutrient fibre material comprises almost an entire insoluble fibre content of sugarcane, comprising a lignose, hemicellulose and cellulose combination, including pectin, xylan and arabinoxylan polymers.

37. The method of claim 30, wherein the prebiotic phytonutrient fibre material is prepared using a process comprising the steps of: subjecting the sugarcane to at least one wet diffusion step to separate sugars from a residual fibre material whilst maintaining nutrient content; and, subjecting the residual fibre material to a rapid, low-heat drying process to retain biologically active molecules in the residual fibre material and to enhance water retention properties of said residual fibre material, to thereby produce the prebiotic phytonutrient fibre material.

38. A composition comprising a prebiotic phytonutrient fibre material extracted from sugarcane, and at least one probiotic bacterial strain.

39. The composition of claim 38, wherein the composition is in a form selected from the group consisting of a pellet, food product, and beverage.

40. The composition of claim 38, wherein the composition comprises at least one ingredient selected from the group consisting of a pharmaceutically acceptable carrier, solvent, base and excipient.

41. The composition according to claim 38, wherein said at least one probiotic bacterial strain is present as spores.

42. The composition of claim 38, wherein said prebiotic phytonutrient fibre material is sucrose-reduced sugarcane fibre.

43. The composition of claim 38, wherein said prebiotic phytonutrient fibre material comprises almost the entire insoluble fibre content of sugarcane, comprising a lignose, hemicellulose and cellulose combination, including pectin, xylan and arabinoxylan polymers.

44. The composition of claim 38, wherein the prebiotic phytonutrient fibre material is prepared using a process comprising the steps of: subjecting the sugarcane to at least one wet diffusion step to separate sugars from a residual fibre material whilst maintaining nutrient content; and, subjecting the residual fibre material to a rapid, low-heat drying process to retain biologically active molecules in the residual fibre material and to enhance water retention properties of said residual fibre material, to thereby produce the prebiotic phytonutrient fibre material.

45. A composition comprising a prebiotic phytonutrient fibre material extracted from sugarcane, and at least one postbiotic.

46. The composition of claim 45, wherein the composition is in a form selected from the group consisting of a pellet, food product, and beverage.

47. The composition of claim 45, wherein the composition comprises at least one ingredient selected from the group consisting of a pharmaceutically acceptable carrier, solvent, base and excipient.

48. The composition of claim 45, wherein said prebiotic phytonutrient fibre material is sucrose-reduced sugarcane fibre.

49. The composition of claim 45, wherein said prebiotic phytonutrient fibre material comprises almost the entire insoluble fibre content of sugarcane, comprising a lignose, hemicellulose and cellulose combination, including pectin, xylan and arabinoxylan polymers.

50. The composition of claim 45, wherein the prebiotic phytonutrient fibre material is prepared using a process comprising the steps of: subjecting the sugarcane to at least one wet diffusion step to separate sugars from a residual fibre material whilst maintaining nutrient content; and, subjecting the residual fibre material to a rapid, low-heat drying process to retain biologically active molecules in the residual fibre material and to enhance water retention properties of said residual fibre material, to thereby produce the prebiotic phytonutrient fibre material.
